(19) United States
(12) Patent Application Publication
(10) Pub. No.: US 2002/0168739 A1
(43) Pub. Date: Nov. 14, 2002

(54) Vectors, compositions and methods for treating a vascular disorder

(76) Inventor: Kenneth K. Wu, Houston, TX (US)

Correspondence Address: Gilbreth & Associates, P.C., PO Box 2428, Bellaire, TX, 77402-2428, US

(21) Appl. No.: 10/094,210

(22) Filed: Mar. 8, 2002

Related U.S. Application Data
(60) Provisional application No. 60/274,866, filed on Mar. 9, 2001.

(51) Int. Cl. C12P 7/40; C12N 9/02; C12N 15/867; C12N 15/861; A61K 48/00

(52) U.S. Cl. 435/189; 435/456; 435/320.1; 435/136; 514/44

(57) ABSTRACT

The present invention discloses vectors comprising a cyclooxygenase sequence, a prostaglandin synthase sequence or both. The invention further discloses methods of making such vectors, and compositions comprising such vectors. Methods for treating a patient afflicted with a vascular disorder by use of said vectors and compositions are also disclosed.

### REFERENCE TO GOVERNMENT FUNDING

The present invention was supported by grants R01-HL-50675 and P50-NS-23327 awarded by the National Institutes of Health.

### BACKGROUND OF THE INVENTION

1. Field of the Invention

The present invention relates to expression vectors, gene transfer, and methods of increasing vascular and tissue protection. In another aspect, the present invention relates to expression vectors comprising a cyclooxygenase gene sequence, vectors comprising a prostaglandin synthase gene sequence, and vectors comprising both a cyclooxygenase gene sequence and a prostaglandin synthase gene sequence. In even another aspect, the present invention relates to compositions comprising a vector comprising either a cyclooxygenase gene sequence, a prostaglandin synthase gene sequence, or both. In still another aspect, the present invention relates to compositions and methods for treating a patient afflicted with a vascular disorder.

2. Description of the Related Art

Prostacyclin, also referred to as prostaglandin I₂ (PGI₂), is a vasodilator and a potent inhibitor of platelet aggregation. Among other effects such as, for example, functioning in cell protection in brain cells, PGI₂ acts in concert with nitric oxide, ectonucleotidase and other endothelial molecules to maintain vascular homeostasis and vasoprotection (Wu K K, Thiagarajan P. Role of endothelium in thrombosis and hemostasis. Annu. Rev. Med. 1996;47: 315-331). PGI₂ is synthesized primarily in vascular endothelial and smooth muscle cells following appropriate stimulation by specific agents. The biosynthesis of PGI₂ is catalyzed by a series of enzymes: cytosolic phospholipase A₂ cleaves arachidonic acid (AA) from the sn-2 position of phospholipids, cyclooxygenase (COX) converts AA to PGH₂, and PGI₂ synthase (PGIS) converts PGH₂ to PGI₂ (Wu K K, Kulmacz R J, Wang L-H, et al. Molecular biology of prostacyclin biosynthesis. In Prostacyclin: New perspectives for Basic Research and Novel Therapeutic Indications. G Rubanyi, J R Vane, Editors. Amsterdam, Netherlands: Elsevier Science Publishers BV. 1992; 11-23). PGH₂ is a precursor of several biologically active prostanoids, including prostaglandin E₂ (PGE₂), PGD₂, PGF₂a and thromboxane A₂ (TXA₂). Two COX isoforms, COX-1 and COX-2, have been identified in endothelial cells (EC). COX-1 is expressed constitutively, whereas COX-2, is undetectable in resting cells, and is induced by proinflammatory and mitogenic factors (Wu K K. Inducible cyclooxygenase and nitric oxide synthase. Adv Pharmacol. 1995;33: 179-207, Smith W L, Marnett L J. Prostaglandin endoperoxide synthase: structure and catalysis. Biochem Biophys Acta. 1990;1083: 1-14). COX is considered a key step in determining the capacity for the synthesis of PGI₂ and other PGs (Smith W L, Marnett L J. Prostaglandin endoperoxide synthase: structure and catalysis. Biochem Biophys Acta. 1990;1083: 1-14).

It has been previously shown by retrovirus-mediated gene transfer that overexpression of COX-1 in EC is accompanied by a marked increase in prostanoid synthesis, notably PGI₂ and PGE₂, in response to stimulation by AA, ionophore A23187 or thrombin (Xu X-M, Ohashi K, Sanduja S K, et al. Enhanced prostacyclin synthesis in endothelial cells by retrovirus-mediated transfer of prostaglandin H synthase cDNA. J Clin Invest. 1993; 31: 1843-1849). Similarly, adenovirus-mediated COX-1 gene transfer in EC enhanced the production of PGI₂, and direct administration of adenovirus-COX-1 (Ad-COX-1) into injured porcine carotid arteries abrogated thrombus formation which was determined by histological examinations and flow measurements as a result of increased PGI₂ production by the injured artery (Zoldhelyi P, McNatt J, Xu X-M, et al. Prevention of arterial thrombosis by adenovirus-mediated transfer of cyclooxygenase gene. Circulation. 1996;93: 10-17). The antithrombotic effect depended on the titer of Ad-COX-1 (Zoldhelyi P, McNatt J, Xu X-M, et al. Prevention of arterial thrombosis by adenovirus-mediated transfer of cyclooxygenase gene. Circulation. 1996;93: 10-17). Overexpression of PGIS by gene transfer was reported to increase PGI₂ production and inhibit smooth muscle cell proliferation in a rat carotid artery injury model (Tanaka T, Yokoyama C, Yamamoto H, et al. Gene transfer of human prostacyclin synthase prevents neointimal formation after carotid balloon injury in rats. Stroke. 1999;30: 419-426).

Unfortunately, there are drawbacks associated with each of these methods of enhancing PGI₂ synthesis by overexpression of a single synthetic enzyme such as COX-1, or PGIS. For example, it is known that in cells transfected with a COX-1 gene, in addition to an augmented PGI₂ synthesis, a large quantity of PGE₂ is also produced. PGE₂ is a pro-inflammatory mediator and PGE₂ overproduction is believed to produce undesirable effects (Hinson R M, Williams J A, Schacter E. Elevated interleukin 6 by prostaglandin E₂ in a murine model of inflammation: Possible role of cyclooxygenase 2. PNAS, USA, 1996; 93:4885-4889.).

In spite of advancements in the art, apparatus and methods to increase prostacyclin levels while not increasing levels of other prostanoids such as, for example, prostaglandin E₂, have not been demonstrated. Thus, there remains a need for desirable gene therapy apparatus and methods useful in treating a patient having a vascular disorder, wherein said apparatus and methods increase prostacyclin levels while not increasing levels of other prostanoids.

There is another need in the art for vectors comprising a cyclooxygenase sequence and/or a prostaglandin synthase sequence.

There is even another need in the art for compositions useful in treating a patient afflicted with a vascular disorder, wherein the compositions comprise a cyclooxygenase sequence and/or a prostaglandin synthase gene sequence, and wherein the sequences are nucleic acid or amino acid sequences.

There is still another need in the art methods for treating a patient afflicted with a vascular disorders wherein the methods comprise gene therapy.

These and other needs will become apparent to those of skill in the art upon review of this specification, including its drawings, claims and appendix.

### SUMMARY OF THE INVENTION

It is an object of the present invention to provide gene therapy apparatus and methods useful in treating vascular disorders by increasing prostacyclin levels while not increasing levels of other prostanoids.

It is another object of the present invention to provide vectors comprising a cyclooxygenase sequence and/or a prostaglandin synthase sequence. Preferably the prostaglandin synthase sequence is a prostacyclin sequence.

It is even another object of the present invention to provide compositions useful in treating a patient afflicted with a vascular disorder, wherein the compositions comprise a cyclooxygenase sequence and/or a prostaglandin synthase sequence, and wherein the sequences may be nucleic acid or amino acid sequences. Preferably the cyclooxygenase sequence is a cyclooxygenase-1 sequence, and the prostaglandin synthase sequence is a prostacyclin sequence.

It is still another object of the present invention to provide methods for treating a patient afflicted with a vascular disorder wherein the methods comprise gene therapy.

According to one embodiment of the present invention there is provided a vector comprising either a cyclooxygenase nucleic acid sequence encoding at least a portion of a coding region of a cyclooxygenase gene, a prostaglandin synthase nucleic acid sequence encoding at least a portion of a coding region of a prostaglandin synthase gene, or both. Preferably the cyclooxygenase sequence is a cyclooxygenase-1 sequence, and the prostaglandin synthase sequence is a prostacyclin sequence.

According to another embodiment of the present invention there is provided a composition comprising a vector comprising a cyclooxygenase nucleic acid sequence encoding at least a portion of a coding region of a cyclooxygenase gene, and a prostaglandin synthase nucleic acid sequence encoding at least a portion of a coding region of a prostaglandin synthase gene. Preferably the cyclooxygenase sequence is a cyclooxygenase-1 sequence, and the prostaglandin synthase sequence is a prostacyclin sequence.

According to even another embodiment of the present invention there is provided a composition comprising a cyclooxygenase-1 peptide and a prostaglandin I₂ synthase peptide.

According to still another embodiment of the present invention there is provided a method of making a vector. Generally the method comprising the steps of ligating an expression vector with a cyclooxygenase-1 (COX-1) nucleic acid sequence and a prostaglandin I₂ synthase (PGIS) nucleic acid sequence to produce a bicistronic COX-1-PGIS expression vector.

According to yet another embodiment of the present invention there is provided a method of treating a patient. Generally the method comprises the step of administering to a patient a composition comprising a vector. The vector of the composition comprises a cyclooxygenase nucleic acid sequence encoding at least a portion of a coding region of a cyclooxygenase gene, and a prostaglandin synthase nucleic acid sequence encoding at least a portion of a coding region of a prostaglandin synthase gene. Preferably the cyclooxygenase sequence is a cyclooxygenase-1 sequence, and the prostaglandin synthase sequence is a prostacyclin sequence.

According to even still another embodiment of the present invention there is provided a method for treating a patient. Generally the method comprises the steps of administering to a patient a composition comprising a vector, wherein the vector comprises a cyclooxygenase nucleic acid sequence encoding at least a portion of a coding region of a cyclooxygenase gene. The method further comprises administering to the patient a composition comprising a vector comprising a prostaglandin synthase nucleic acid sequence encoding at least a portion of a coding region of a prostaglandin synthase gene. Preferably the cyclooxygenase sequence is a cyclooxygenase-1 sequence, and the prostaglandin synthase sequence is a prostacyclin sequence.

According to even yet another embodiment of the present invention there is provided a method of treating a patient. Generally the method comprises the steps of increasing the level of prostaglandin I₂ protein in a patient, and increasing the level of cyclooxygenase-1 protein in a patient. Preferably, the level of other prostanoids such as, for example, prostaglandin E₂, in the patient does not increase as a result of steps a and b.

According to still even another embodiment of the present invention there is provided a method of treating a patient. Generally the method comprises the steps of administering to a patient a composition comprising a cyclooxygenase-1 peptide and a prostaglandin I₂ synthase peptide. Preferably the level of other prostanoids such as, for example, prostaglandin E₂ and prostaglandin F_{2α}, in the patient are not increased as a result of the treatment method.

According to still yet another embodiment of the present invention there are provided any and all methods for treating a patient having a vascular disorder, wherein the method comprises increasing the level of prostaglandin I₂ in a patient, and increasing the level of cyclooxygenase-1 in a patient. Preferably the level of other prostanoids such as prostaglandin E₂ and prostaglandin F_{2α}, in the patient are not increased as a result of steps and b.

These and other embodiments of the present invention will become apparent to those of skill in the art upon review of this specification, including its drawings, appendix, and claims.

### BRIEF DESCRIPTION OF THE DRAWINGS

FIG. 1A depicts a construct of a bicistronic pCOX-1/PGIS plasmid of the invention.

FIG. 1B shows the results of a Western blot analysis using extract from ECV304 cells transfected with a bicistronic pCOX-1/PGIS plasmid, the control plasmid pCOX-1, or the control plasmid PPGIS of the invention.

FIG. 2 shows the results from an HPLC analysis of [1-¹⁴C]AA metabolites.

FIG. 3 shows the results from a Western blot analysis of COX-1 and PGIS protein expression in human umbilical vein endothelial cells (HUVECs) transfected with various m.o.i. ratios of Ad-COX-1 and Ad-PGIS.

FIG. 4 shows the results from an analysis of eicosanoids generated by transfected HUVECs in response to [1-¹⁴C]AA treatment.

FIG. 5 illustrates the synergy resulting from gene co-transfer of cyclooxygenase-1 and prostacyclin synthase into rabbit vascular smooth muscle cells.

FIG. 6. illustrates the effect of Adv.hPGK and Adv.hPGK-COX-1-PGIS infusion on the cerebral infarct volume after 60 minute transient ischemia.

FIG. 7. illustrates the effect of Adv.hPGK or Adv.hPGK-COX-1-PGIS infusion on cortical Pgs and Lts concentration after 60 minute transient cerebral ischemia.

### DETAILED DESCRIPTION OF THE INVENTION

The present invention is directed to vectors comprising a cyclooxygenase sequence, a prostaglandin synthase sequence, or both a cyclooxygenase sequence and a prostaglandin synthase sequence. The invention is also directed to methods of making such vectors, and to compositions and methods utilizing such vectors. While the cyclooxygenase and prostaglandin synthase sequences used herein may be any cyclooxygenase and prostaglandin synthase sequence, it is generally preferred to utilize a cyclooxygenase-1 sequence, and a prostaglandin I₂ synthase sequence. Prostaglandin I₂ synthase (PGIS) is also referred to as prostacyclin.

According to one embodiment of the invention there is provided a bicistronic vector comprising a cyclooxygenase nucleic acid sequence encoding at least a portion of a coding region of a cyclooxygenase gene, and a prostaglandin synthase nucleic acid sequence encoding at least a portion of a coding region of a prostaglandin synthase gene. In a preferred embodiment, the cyclooxygenase gene is cyclooxygenase-1 and the prostaglandin synthase gene in prostaglandin I₂ synthase.

The nucleic acid sequences utilized in the invention are not size restricted. Generally the cyclooxygenase sequence is a full-length sequence, such as a full-length cDNA sequence, and the prostaglandin I₂ synthase sequence is a full-length sequence, such as a full-length PGIS cDNA sequence. However, it is possible to use any portion of a cyclooxygenase sequence and any portion of a prostaglandin I₂ synthase sequence as long as the sequences produce the result desired in the present invention.

The cyclooxygenase and prostaglandin synthase gene sequences of the invention may be sequences from any organism, such as, for example, mouse, sheep, cow, pig, and human sequences. In a preferred embodiment, the cyclooxygenase nucleic acid sequence and the prostaglandin synthase nucleic acid sequence used in the present invention are mammalian sequences. In a more preferred embodiment, the cyclooxygenase and prostaglandin synthase gene sequences used in the present invention are human cyclooxygenase-1 and human prostaglandin I₂ synthase gene sequences. These sequences are known in the art, such as those found in GenBank.

Another embodiment of the invention is directed to a method of making a bicistronic vector. Generally the method comprises the steps of ligating an empty expression vector with a cyclooxygenase-1 (COX-1) nucleic acid sequence and a prostaglandin I₂ synthase (PGIS) nucleic acid sequence to produce a COX-1-PGIS expression vector. Ligation techniques are well known to those of skill in the art, and all such techniques are applicable herein. Preferably, construction of the COX-1-PGIS expression vector results in the cyclooxygenase-1 nucleic acid and the prostaglandin I₂ synthase nucleic acid each being operatively linked to regulatory sequences which direct the expression of said cyclooxygenase-1 and prostaglandin I₂ synthase sequences.

The vectors utilized herein may be any expression vector known in the art. Suitable expression vectors known in the art include bacterial vectors, viral vectors, and eukaryotic vectors such as, for example, yeast vectors and mammalian vectors. In principle, all vectors which replicate and express the desired sequence according to the invention in the chosen host are suitable. Thus, the vector may be a plasmid such as, for example, pSG5, or the vector may be a viral vector such as, for example, a retroviral vector, an adenoassociated vector, an adenoviral vector, a lentiviral vector, or a herpes viral vector. A preferred viral vector in the present invention is an adeonoassociated viral vector or an adenoviral vector such as, for example, the adenovirus shuttle plasmid vector Ad-CMV, and Ad-PKG.

Even another embodiment of the invention is directed to a composition. The compositions of the invention generally comprise a vector comprising a cyclooxygenase nucleic acid sequence encoding at least a portion of a coding region of a cyclooxygenase gene, and a vector comprising a prostaglandin synthase nucleic acid sequence encoding at least a portion of a coding region of a prostaglandin synthase gene. The cyclooxygenase nucleic acid sequence and the prostaglandin synthase nucleic acid sequence may be expressed from a single vector wherein the vector is a bicistronic vector, or they may each be expressed from separate and distinct vectors. In a preferred embodiment, the nucleic acid sequences are expressed from a bicistronic vector comprising both a cyclooxygenase nucleic acid sequence and a prostaglandin synthase nucleic acid sequence. The compositions of the invention are useful in treating a patient having a vascular disorder.

Alternatively, the compositions of the invention may comprise a cyclooxygenase-l peptide, a prostaglandin I₂ synthase peptide, or both. Techniques for expressing and purifying recombinant proteins and peptides are well known by those of skill in the art, and all such techniques, for example, those discussed in S. Hornemann et al., (1997), FEBS Lett. 413, 277-281; Maniatis T. et al., “Molecular Cloning, a Laboratory Manual”, Cold Spring Harbor Laboratory, Cold Spring Harbor, N.Y., 1982; Ausubel F. M. et al. (eds); and “Current Protocols in Molecular Biology”, John Wiley & Sons, New York, (1987) are applicable and are incorporated herein by reference.

The preparation of compositions is also well known in the art, and all such techniques are appropriate in preparing the compositions of the present invention, and are incorporated herein by reference. The compositions of the present invention further comprise a pharmaceutically acceptable carrier/vehicle. Pharmaceutically acceptable carriers/vehicles are known in the art and include aqueous solutions, non-toxic excipients, including salts, preservatives, buffers and the like, propylene glycol, polyethylene glycol, vegetable oil, injectable organic esters such as ethyloleate, water, saline solutions, parenteral vehicles such as sodium chloride and Ringer's dextrose, glycerol, lipids, alcohols.

Compositions of the present invention may be in any form known in the art, such as an orally digestible form, a sterile injectable form, forms suitable for delayed release, and forms that are enterically coated. Compositions of the invention may be in solid forms, including, for example, powders, tablets, pills, granules, capsules, sachets and suppositories, or may be in liquid forms including solutions, suspensions, gels and emulsions. Preferably, the composition of the invention is in a liquid form such as, for example, a solution, suspension or emulsion.

Still another embodiment of the invention is directed to a method of treating a patient. Generally the treatment method comprises the step of administering to a patient a composition comprising a bicistronic vector of the invention. The bicistronic vector comprises a cyclooxygenase nucleic acid sequence encoding at least a portion of a coding region of a cyclooxygenase gene, and a prostaglandin synthase nucleic acid sequence encoding at least a portion of a coding region of a prostaglandin synthase gene.

As used herein, the word “patient” includes any and all organisms capable of developing a vascular disorder. Preferably, the patient of the invention is a mammal. In a particularly preferred embodiment, the patient is a human.

Yet another embodiment of the invention is directed to a method for treating a patient wherein the method comprises the steps of administering to a patient a composition comprising a vector comprising a cyclooxygenase nucleic acid sequence encoding at least a portion of a coding region of a cyclooxygenase gene; and administering to the patient a composition comprising a vector comprising a prostaglandin synthase nucleic acid sequence encoding at least a portion of a coding region of a prostaglandin synthase gene. In this method of the invention, step a may be carried out before, after, or at about the same time as step b.

Even still another embodiment of the invention is directed to a method of treating a patient. Generally the method comprises the steps of increasing the level of prostaglandin I₂(PGI₂) in a patient, and increasing the level of cyclooxygenase-1 protein in a patient. Preferably the level of other prostanoids such as prostaglandin E₂, in the patient does not increase as a result of treatment.

Even yet another embodiment of the invention is directed to a method of treating a patient. Generally the method comprises the steps of administering to a patient a composition comprising a cyclooxygenase-1 peptide and a prostaglandin I₂ synthase (PGIS) peptide wherein the level of prostanoids such as, for example, prostaglandin E₂ and prostaglandin F_{2α}, in the patient are not increased.

Still even another embodiment of the invention is directed to a method of treating a patient wherein the method comprises the steps of administering to a patient a composition comprising a cyclooxygenase-1 peptide and a prostaglandin I₂ synthase peptide. As with the other treatment methods of the present invention, preferably the level of other prostanoids such as, for example, prostaglandin E₂ and prostaglandin F_{2α}, in the patient are not increased as a result of the treatment method.

The vectors, compositions and methods of the invention are useful for treating a patient afflicted with a vascular disorder. The disorder may be in any stage of progression. Preferably the disorder is associated with at least one condition selected from the group consisting of stroke, pulmonary hypertension, coronary artery disease, cerebrovascular thrombosis, myocardial infarction, diabetic peripheral vascular disease, and non-diabetic peripheral vascular disease. The condition associated with the vascular disorder may be in any stage of development.

In general, the apparatus, compositions and methods of the present invention provide for increased production of a cyclooxygenase gene product, such as cyclooxygenase-1 (COX-1), and a prostaglandin synthase gene product, such as prostaglandin I₂ synthase (PGIS), also referred to as prostacyclin synthase. The apparatus, compositions and methods of the present invention preferably provide overexpression of cyclooxygenase-1 (COX-1) and prostaglandin I₂ synthase (PGIS) at a cyclooxygenase-1/prostaglandin I₂ synthase ratio of about 6, more preferably at a ratio of about 2, most preferably at a ratio of about 1. Preferably, the ratio of COX-1 to PGIS may be any ratio that results in an increase in prostacyclin levels while not increasing the levels of other prostanoids such as prostaglandin E₂ (also referred to as PGE₂), the optimal ratio being that ratio which has the greatest effect. It is possible that the optimal ratio will vary depending upon the vectors, the COX-1 and PGIS sequences utilized, the compositions, and modes of administration utilized, as well as from patient to patient.

If retroviral-mediated infection is used as the mode of administering the invention to a patient, it is generally preferred to utilize a ratio of retroviral-COX-1 m.o.i. to retroviral-PGIS m.o.i. wherein the amount of retroviral-COX-1 is in the range of about 1 to about 6 m.o.i., and the retroviral-PGIS is in the range of about 0.5 to about 2 m.o.i.

As one example, using retrovirus-mediated infection as a mode of administering the invention to human umbilical vein endothelial cells (HUVECs), it is generally preferred to select an Ad-COX-1 to Ad-PGIS ratio of at least about 50 to about 100 Ad-COX-1 m.o.i. to about 50 Ad-PGIS m.o.i.

The administration of the compositions of the present invention may be systemic or localized. Administration may be by any method known in the art. Thus, administration of the present invention to a recipient/patient may be by a route selected from inhalation, oral, parenteral (including, subcutaneous, intradermal, intramuscular, intra-cerebral ventricle, and intravenous) and rectal. For increased efficacy, the compositions of the present invention may be administered via localized delivery to a targeted region or tissue. Preferably the compositions of the present invention are administered by direct intra-arterial or intravenous injection, or by injection into at least one targeted site within the patient such as, for example, the brain, a cerebral ventricle of the brain, the heart, a femoral artery, a coronary artery, and any ischemic tissue. A particularly preferred mode of administering the invention to a patient who has had a stroke, is by direct injection into a cerebral ventricle. For patients suffering from pulmonary hypertension, a preferred mode of administering the invention is via inhalation. For patients suffering from other vascular disease, the invention may be delivered locally by infusion through femoral arteries, or retaining vectors in a segment of coronary arteries.

The compositions and methods of the present invention may be administered to a recipient/patient as a single dose unit, or may be administered in several dose units, for a period ranging from one day to several years. The dose schedule is dependent upon at least the severity of the patient's disorder, as well as the composition and mode of administration.

Still yet another embodiment of the invention is directed to any and all methods for treating a patient afflicted with a vascular disorder in any stage of progression, wherein the method comprises enhancing the patient's level of prostacyclin without enhancing the patient's level of other prostanoids such as prostaglandin E₂ or F_{2α}.

All references cited in the present application, including journal articles, laboratory manuals, all U.S. and foreign patents and patent applications, are specifically and entirely incorporated by reference.

### EXAMPLES

The following examples are provided to illustrate the present invention. These examples are not intended to limit the scope of the claims of the present invention, and should not be so interpreted.

### Example 1

### Recombinant Plasmid Construction

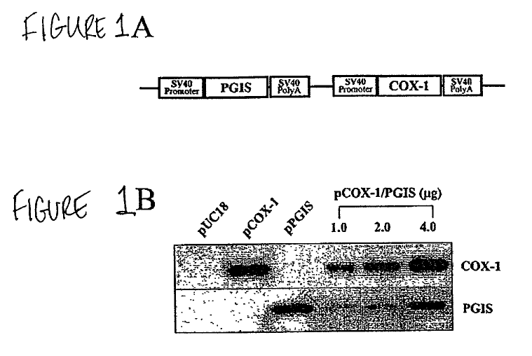

pCOX-1 and pPGIS were constructed in pSG5 as previously described (6, 15). A bicistronic pCOX-1/PGIS was constructed in pSG5 by SalI digestion to remove the COX-1 expression cassette, and subcloned into the NdeI site of pPGIS. The final construct contains two expression cassettes which are driven by independent SV40 promoters, as shown in FIG. 1A.

### Example 2

### Cell Culture and Materials

ECV304 were used in the present transfection examples prior to the report that they exhibit endothelial, epithelial and bladder cancer cell characteristics (Kiessling F, Kartenbeck J, Haller C. Cell-cell contacts in human cell line ECV304 exhibit both endothelial and epithelial characteristics. Cell & Tissue Res. 1999;297: 131-140). The ECV304 cells used in the present examples stain positively for von Willebrand factor, are capable of expressing adenovirus-transferred COX-1 and PGIS transgenes which colocalize to endoplasmic reticulum (ER) as the natively expressed enzymes, and have a large prostacyclin synthetic capacity (Liou J-Y, Shyue S-K, Tsai M-J, et al. Colocalization of prostacyclin synthase with prostaglandin H synthase-1 but not phorbol ester-induced PGHS-2 in cultured endothelial cells. J Biol Chem. 2000;275: 15314-15320). Thus, ECV304 retains EC properties.

ECV304 and 293 cells were obtained from the American Type Culture Collection. The cells were maintained in Dulbecco's modified Eagle's medium (DMEM) supplemented with 10% fetal bovine serum (FBS), 100 U/ml penicillin and 100 mg/ml streptomycin at 370 in a humidified 5% CO₂ atmosphere. Human umbilical vein endothelial cells (HUVECs) were prepared from freshly obtained umbilical veins and cultured as previously described (Xu X-M, Ohashi K, Sanduja S K, et al. Enhanced prostacyclin synthesis in endothelial cells by retrovirus-mediated transfer of prostaglandin H synthase cDNA. J Clin Invest. 1993;31: 1843-1849). HUVECs used were from passages 3 to 6. Although COX-1 expressions decline with increasing HUVEC passages, HUVECs from passage 3 to 6 are capable of synthesizing prostanoids which are quantitatively reduced but qualitatively similar to cells at earlier passages. Furthermore, passage 3-6 HUVECs expressed adenovirus-mediated COX-1 and PGIS transgenes competently as demonstrated by confocal immunofluorescent microscopy (Liou J-Y, Shyue S-K, Tsai M-J, et al. Colocalization of prostacyclin synthase with prostaglandin H synthase-1 but not phorbol ester-induced PGHS-2 in cultured endothelial cells. J Biol Chem. 2000;275: 15314-15320). There was no significant difference in transfection results among cells at passages 3-6. Cell culture media and antibiotics were obtained from BRL Life Technologies. [1-¹⁴C]-AA (55 mCi/mmol) was from Amersham.

FIG. 1B shows that basal COX-1 and PGIS protein levels are low in non-transfected or pUC18 (negative control) transfected ECV304 cells. FIG. 1B further shows ECV304 cells exhibit a concentration-dependent increase in COX-1 and PGIS levels when the cells are transfected with 1, 2, and 4 μg of bicistronic pCOX-1/PGIS vector.

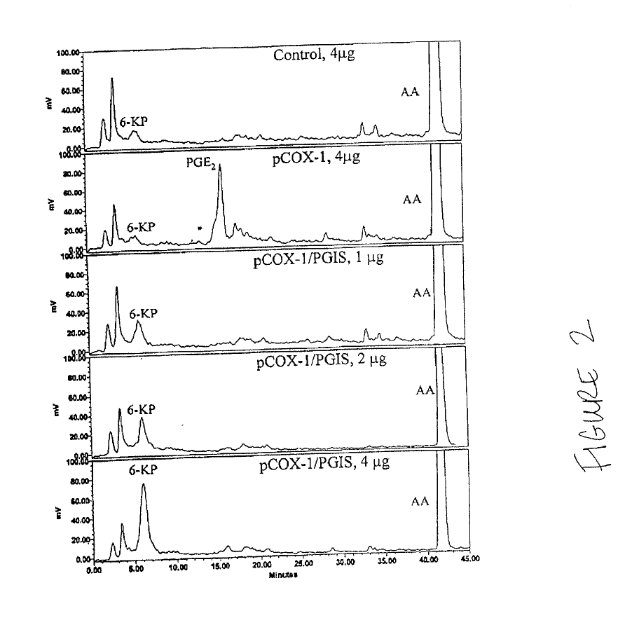

FIG. 1B, FIG. 2, and Table 1 show that the COX-1 protein level in ECV304 cells transfected with 4 μg of pCOX-1/PGIS is comparable to that of ECV304 cells transfected with 4 μg of pCOX-l, while the PGIS protein level in pCOX-1/PGIS transfected cells is only about 50% of that in pPGIS transfected cells (FIG. 1B).

TABLE 1
Quantitation of Major Prostanoid Peaks
Produced By ECV304 Cells as Shown in FIG. 2.

| | 6-keto-PGF_{1α} | PGE₂ | HHT |
|---|---|---|---|
| Plasmids | (ng) | (ng) | (ng) |
| Control | 2.88 | 0.20 | 0.00 |
| pCOX-1, | 2.43 | 25.79 | 1.03 |
| 4 μg | | | |
| pCOX-1/PGIS, | 10.22 | 0.16 | 0.53 |
| 1 μg | | | |
| pCOX-1/PGIS, | 11.92 | 0.38 | 0.10 |
| 2 μg | | | |
| pCOX-1/PGIS, | 30.45 | 1.44 | 0.72 |
| 4 μg | | | |

FIG. 2 and Table 1 further show a marked difference in the HPLC profile of cells transfected with bicistronic pCOX-1/PGIS vs. pCOX-1 or pPGIS. Transfection of cells with pCOX-1 (4 μg) increases PGE₂ synthesis by about 100-fold over the control level (FIG. 2 and Table 1). In contrast, transfection with bicistronic pCOX-1/PGIS increases 6-keto-PGF₁ₐ levels in a concentration-dependent manner without a concurrent PGE₂ increase (FIG. 2 and Table 1). The 6-keto-PGF₁ₐ level produced by cells transfected with 4 μg of bicistronic pCOX-1/PGIS is about 12-fold higher than that produced by cells transfected with 4 μg of pCOX-1 (FIG. 2 and Table 1). HHT productions are increased in cells transfected with pCOX-1 and bicistronic pCOX-1/PGIS. Although not intending to be bound or limited by theory, the applicant suggests it is possible that co-overexpression of COX-1 and PGIS re-directs PGH₂ through the PGIS pathway.

### Example 3

### Recombinant Adenovirus Production

Replication-defective adenoviruses were produced as described (Zoldhelyi P, McNatt J, Xu X-M, et al. Prevention of arterial thrombosis by adenovirus-mediated transfer of cyclooxygenase gene. Circulation. 1996;93: 10-17; Gomez-Foix A M, Coats W S, Baque S, et al. Adenovirus-mediated transfer of the muscle glycogen phosphorylase gene into hepatocytes confers altered regulation of glycogen metabolism. J Biol Chem. 1992;267: 25129-25134; Herz J, Gerard R D. Adenovirus-mediated transfer of low-density lipoprotein receptor gene acutely accelerates cholesterol clearance in normal mice. Proc Natl Acad Sci USA. 1993;980: 2812-2816). The adenovirus shuttle plasmid vector pAd-CMV, kindly supplied by Dr. S. -H. Chen at Mount Sinai School of Medicine, contains a CMV promoter and a polyadenylation signal of bovine growth hormone. The recombinant adenovirus (rAd) was prepared by co-transfecting 293 cells with pAd-CMV containing the candidate cDNAs in expression cassettes and pJM17, kindly provided by Dr. L. Chan at Baylor College of Medicine, using an Effectene (Qiagen) transfection system. Two to three weeks after transfection, rAd plaques were picked, propagated and screened for specific cDNA sequence by PCR and protein expression by Western blot analysis.

A large-scale production of high titer rAd was performed as described (Zoldhelyi P, McNatt J, Xu X-M, et al. Prevention of arterial thrombosis by adenovirus-mediated transfer of cyclooxygenase gene. Circulation. 1996;93: 10-17) with minor modifications. 293 cells grown to about 95% confluence were infected with rAd for 36-44 hours, harvested by centrifugation and resuspended in fresh culture medium. After freezing and thawing the cells five times, cells were centrifuged to remove cell debris. The supernatant was collected and rAd harvested by CsCl gradient ultracentrifugation. The opalescent band containing viral particles was collected, loaded onto the top of 1.33 g/ml CsCl, and centrifuged again at the same condition for 18 h. The opalescent band recovered was dialyzed three times against one liter of buffer containing 10 mM Tris pH 7.4, 1 mM MgCl₂ and 10% (v/v) glycerol at 4° C. for 18 h. Virus stocks were aliquoted and stored at −80° C.

Viral titers were determined by a plaque-assay method. 293 cells were infected with serially diluted viral preparations and then overlaid with low melting-point agarose after infection. The numbers of plaques formed were counted within two weeks. Plaque forming units per cell are referred to as multiplicity of infection (m.o.i.). Human umbilical vein endothelial cells (HUVECs) were then transfected with a mixture of Ad-COX-1 and Ad-PGIS using different pfu ratios in order to determine whether the relative quantities of prostanoids produced are influenced by different ratios of COX-1 overexpression to PGIS overexpression.

HUVECs were transfected with either: 1) 50 m.o.i. (pfu/cell) of Ad-COX-1 alone; 2) 50 m.o.i. (pfu/cell) of Ad-PGIS alone; 3) a fixed 50 m.o.i. of Ad-COX-1 together with 10-100 m.o.i. of Ad-PGIS; or 4) a fixed 50 m.o.i. of Ad-PGIS together with 10-100 m.o.i. of Ad-COX-1. The transfected cells were lysed and COX-1 and PGIS protein levels were determined by Western blot analysis.

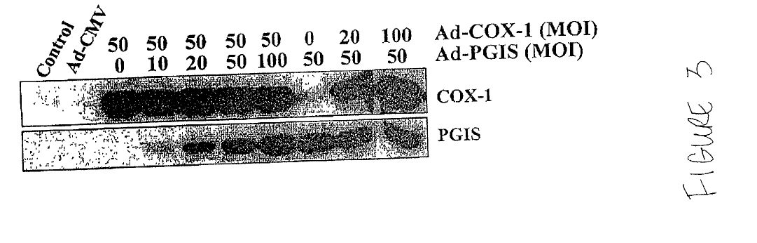

As shown in FIG. 3, individual transfection of Ad-COX-1 and individual transfection of Ad-PGIS produces a marked elevation in COX-1 protein levels and PGIS protein levels, respectively, as compared to non-transfected or Ad-CMV controls. Co-transfection of Ad-COX-1 together with Ad-PGIS produces COX-1 protein levels and PGIS protein levels similar to those produced by transfection of Ad-COX-1 alone or Ad-PGIS alone.

### Example 4

### Extraction and Analysis of AA Metabolites

ECV304 cells were transfected with recombinant plasmids by lipofectamine (Gibco). Forty-eight hours after plasmid transfection and 24 hours after rAd infection, cells were washed and incubated in serum-free DMEM containing 10 μM [1-¹⁴C] AA at 37° C. for 10 min. The media were collected and eicosanoids in the media extracted by Sep-Pak Cartridge (Waters Associates) as previously described (Eling T, Tamer B, Ally A, et al. Separation of arachidonic acid metabolites by high-pressure liquid chromatography. In Methods in Enzymology. WEM Lands and W L Smith, Editors. San Diego, Calif., Academic Press, Inc., 1983; 511-517). The extracted eicosanoids were analyzed by a reverse-phase high pressure liquid chromatography (HPLC) as previously described (Sanduja S K, Mehta K, Xu X-M, et al. Differentiation-associated expression of prostaglandin H and thromboxane A synthase in monocytoid leukemia cell lines. Blood. 1991;78: 3178-3185). The eicosanoid peaks were identified by the retention time of the authentic radiolabeled standards. The quantity of each eicosanoid peak was determined by relating the integrated area (mV*sec) of the peak to the standard obtained from authentic radiolabeled eicosanoids and AA. A 1000 mV*sec integrated area was equivalent to 6.18 ng of AA, 7.44 ng of 6-keto-PGF₁ₐ, 7.12 ng of PGE₂, 7.16 ng of PGF₂ₐ and 5.66 ng of HHT, respectively.

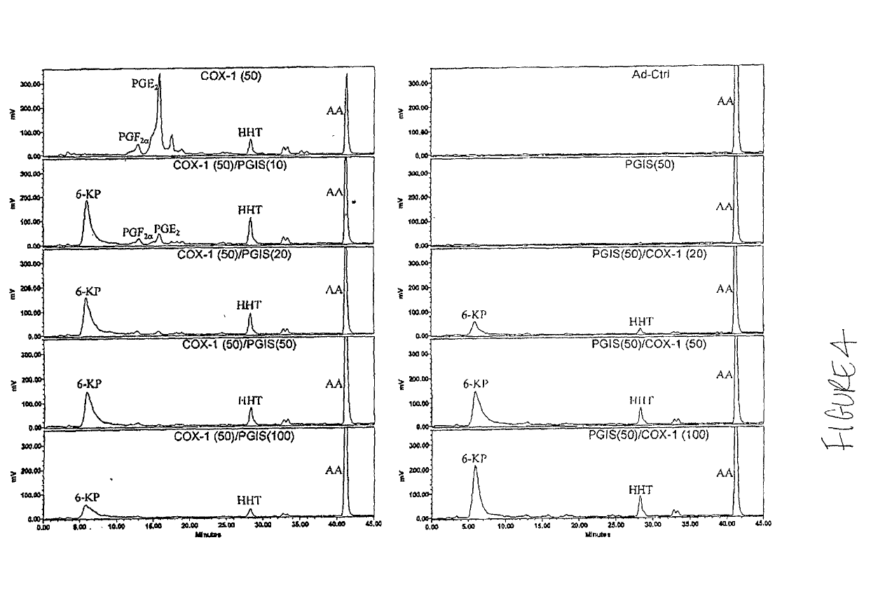

FIG. 4 shows the eicosanoids generated by transfected cells treated with 10 mM [1-¹⁴C]AA. The left panel of FIG. 4 shows the eicosanoid profile of cells co-transfected with Ad-COX-1 at a fixed m.o.i. of 50, together with Ad-PGIS at various m.o.i. of 0-100 m.o.i. The right panel of FIG. 4 shows the profile of cells co-transfected with Ad-PGIS at a fixed m.o.i. of 50 m.o.i., together with Ad-COX-1 at various m.o.i. of 0-100 m.o.i. The right panel of FIG. 4 also shows the profile for the control vector. Table 2 provides the quantitative data for the major prostanoid peaks of FIG. 4.

TABLE 2
Quantitation of Major Prostariold Peaks
Produced by HUVECs as shown in FIG. 4.

| COX1/PGIS* | 6-keto-PGF_{1α} | PGF_{2α} | PGE₂ | HHT |
|---|---|---|---|---|
| (m.o.i.) | (ng) | (ng) | (ng) | (ng) |
| Control | 0.38 | 1.34 | 0.28 | 0.48 |
| 50/0 | 0.85 | 17.9 | 106.4 | 11.20 |
| 50/10 | 96.43 | 7.02 | 10.96 | 18.86 |
| 50/20 | 86.24 | 2.43 | 2.93 | 15.86 |
| 50/50 | 90.76 | 2.43 | 1.21 | 13.29 |
| 100/50 | 105.32 | 1.58 | 0.98 | 15.75 |
| 50/100 | 39.67 | 1.48 | 0.00 | 6.42 |
| 20/50 | 26.58 | 1.51 | 0.20 | 4.71 |
| 0/50 | 2.03 | 0.00 | 0.00 | 0.25 |

(*Ad-COX-1 to Ad-PGIS m.o.i. ratios)

As shown in FIG. 4 and Table 2, of the total prostanoids produced by HUVECs transfected with Ad-COX-1 (50 m.o.i.) alone, PGE₂ is the major product and constitutes 78%, PGF₂ₐ constitutes 13%, and 6-keto-PGF₁ₐ constitutes less than 1%. In contrast, 6-keto-PGF₁ₐ is the predominant product in cells co-transfected with Ad-COX-1/Ad-PGIS. Co-transfection of HUVEC with 100 m.o.i. of Ad-COX-1 and 50 m.o.i. of Ad-PGIS yields the highest 6-keto-PGF₁ₐ levels of 85% of total prostanoids. When the Ad-PGIS m.o.i. is in excess of the Ad-COX-1 m.o.i., the level of 6-keto-PGF₁ₐ as well as the overall level of total prostanoids, are markedly reduced. For example, the 6-keto-PGF₁ₐ and the total prostanoid levels produced by cells transfected with 50 m.o.i. of Ad-COX-1 plus 100 m.o.i. of Ad-PGIS are only 44% and 40%, respectively, of those levels produced by cells transfected with 50 m.o.i. of Ad-COX-1 and 50 m.o.i. of Ad-PGIS (see Table 2).

Also shown in FIG. 4 and Table 2, HHT levels are increased by Ad-COX-1 transfection as well as by Ad-COX-1/Ad-PGIS co-transfection. HHT levels are not increased by Ad-PGIS transfection. The highest level of HHT is produced by cells co-transfected with Ad-COX-1/Ad-PGIS in a ratio of 50/10, or a ratio of 50/20 ratio. HHT levels are reduced when the Ad-PGIS titer is in excess of the Ad-COX-1. Only trace amounts of hydroxyeicosatetraenoic acid (HETE)-like eicosanoids are detected and their values do not detectably vary as a result of the co-transfections.

### Example 5

### Western Blot Analysis

Fifteen μg of cell lysate proteins were applied to each lane and analyzed by Western blots as previously described (Wu K K, Sanduja R, Tsai A-L, et al. Aspirin inhibits interleukin 1-induced prostaglandin H synthase expression in cultured endothelial cells. Proc Natl Acad Sci USA. 1991;88: 2384-2387). PGIS antibodies (Lin Y-Z, Wu K K, Ruan K-H. Characterization of the secondary structure and membrane interaction of the putative membrane anchor domains of PGI synthase and cytochrome P4502C1. Arch Biochem Biophys. 1998;352: 78-84) and COX-1 antibodies (Santa Cruz) were each diluted to 1:2000. Peroxidase-conjugated anti-rabbit or anti-mouse IgG (1:2000 dilution) was used respectively as the second antibody for detection of PGIS and COX-1 bands by ECL (Amersham).

### Example 6

### Gene Transfer of Cyclooxygenase-1 and Prostacyclin Synthase in Cultured Rabbit Vascular Smooth Muscle Cells

Rabbit vascular smooth muscle cells were cultured from rabbit aortic tissue by an explant procedure. Cultured cells were transfected with control adenoviral vectors containing a human PGK promoter (Ad-PGK-null) or a CMV promoter (Ad-CMV-RR), Ad-CMV-COX-1 (300 moi), Ad-PGK-PGIS (300 moi), or combined Ad-CMV-COX-1 (200 moi) and Ad-PGK-PGIS (100 moi). PGI₂ production by the transfected cells was measured as 6-keto-PGF_{1α} by enzyme-immunoassay.

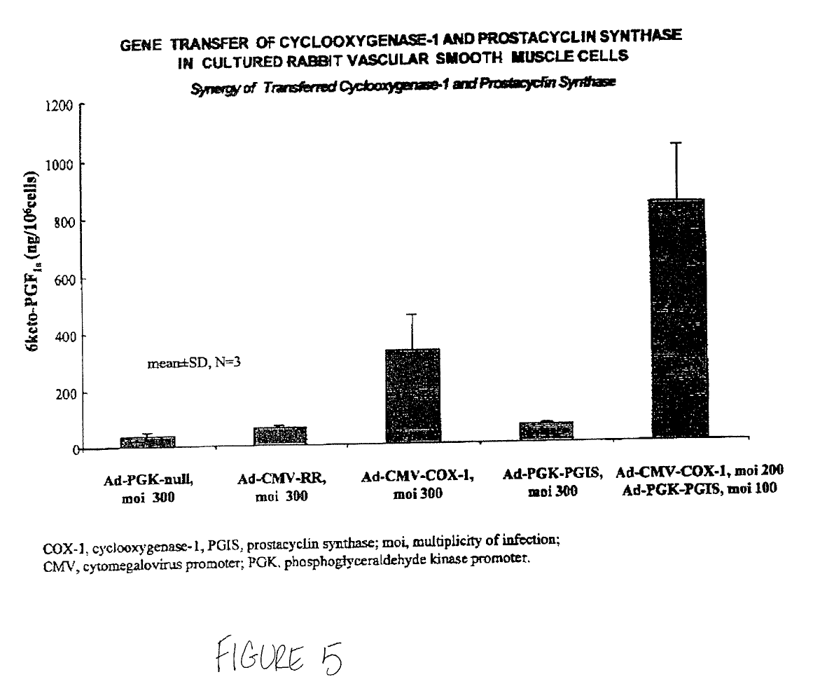

As shown in FIG. 5, combined COX-1 and PGIS gene transfer in endothelial and smooth muscle cells selectively increases PGI₂ production without a concurrent increase in production of other potentially damaging prostaglandins.

### Example 7

### Effect of Adv.hPGK and Adv.hPGK-COX-1-PGIS Infusion on Infarct Volume

The experiments of the present example were carried out using a rat focal cerebral ischemia-reperfusion model. The mid-cerebral arteries were reversibly ligated for 60 minutes and the ligation was released for 60 minutes. The brain infarct volume was measured according to a standard procedure previously described (S I Chi, et al., Neuroscience, 3:475-484). Adenoviral vectors containing a human PGK promoter (hPGK control), a human PGK promoter with COX-1 and PGIS (cop) were injected into the brain ventricle 3 days before ischemia (3D-1S), 1 day before injury (1D-1S), at the same time of ischemic (1S-0), 5 hours after ischemic (1S-5h) and 1 day after ischemia (1S-1D). The data show a marked reduction in infarct volume by Ad-COP injection 3 days, 1 day, same time, and 5 hours after ischemic. However, Ad-COP injected 1 day after ischemic is ineffective. These results support the notion that Ad-COP is an effective treatment for stroke.

### Example 8

### Effect of Adv.hPGK or Adv.hPGK-COX-1-PGIS Pre-Infusion on Cortical PGs and LTs Concentration After Ischemia

After transfection with Ad-hPGK control or Ad-hPGK-COX-1-PGIS (Ad-COP), brain infarct tissues were obtained and the PGE₂, 6-keto-PGF_{1α} (PGI₂), TXB₂, PGD₂, LTB₄, LTC₄ levels in the brain tissue were measured using enzyme-immunoassay. These results show that Ad-COP injection into ventricles increased 6-keto-PGF_{1α} (PGI₂) while reducing PGE₂, PGD₂, LTB₄, LTC₄, and TXB₂. These data support a model in which selective increase of PGI₂ reduces cerebral infarct volume.

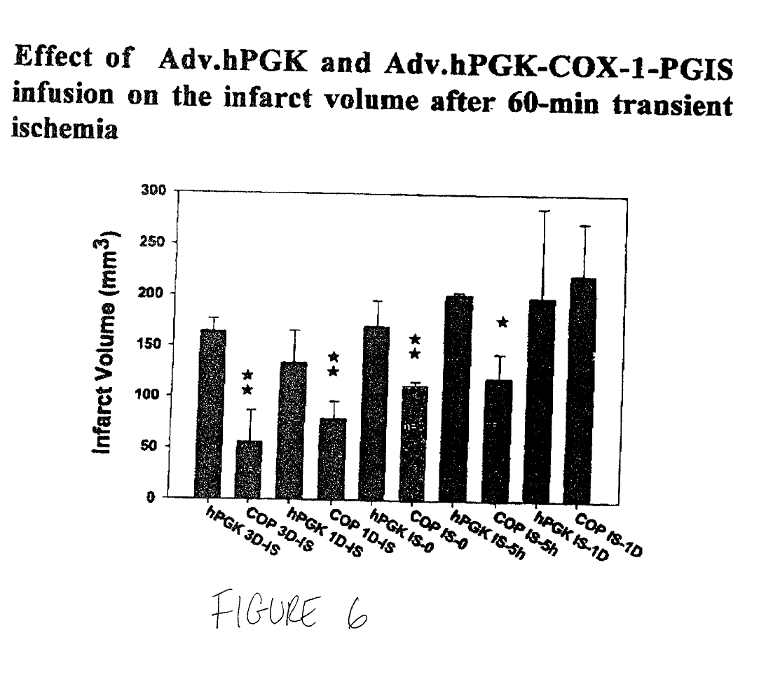

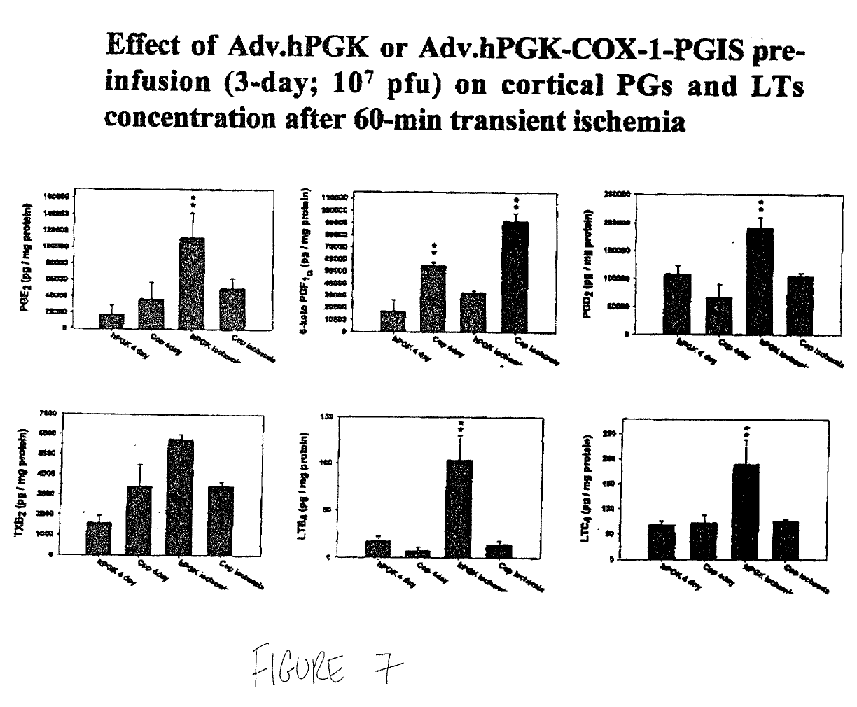

As shown in FIG. 6 and FIG. 7, using rat stroke models, combined COX-1 and PGIS gene transfer increase PGI₂ production in ischemic tissues and causes a significant reduction of brain infarct size 5 hours after ischemic has occurred.

While the illustrative embodiments of the invention have been described with particularity, it will be understood that various other modifications will be apparent to and can be readily made by those skilled in the art without departing from the spirit and scope of the invention. Accordingly, it is not intended that the scope of the claims appended hereto be limited to the examples and descriptions set forth herein, but rather that the claims be construed as encompassing all the features of patentable novelty which reside in the present invention, including all features which would be treated as equivalents thereof by those skilled in the art to which this invention pertains.

We claim:

1. A vector comprising a cyclooxygenase nucleic acid sequence encoding at least a portion of a coding region of a cyclooxygenase gene, and a prostaglandin synthase nucleic acid sequence encoding at least a portion of a coding region of a prostaglandin synthase gene.

2. The vector of claim 1 wherein the cyclooxygenase gene is cyclooxygenase-1 and the prostaglandin synthase gene is prostaglandin I₂ synthase.

3. The vector of claim 1 wherein the vector is a plasmid.

4. The vector of claim 1 wherein the vector is a viral vector.

5. The vector of claim 4 wherein the vector is a retroviral vector, an adenoassociated vector, an adenoviral vector, a lentiviral vector, or a herpes viral vector.

6. The vector of claim 1 wherein the cyclooxygenase nucleic acid sequence and the prostaglandin synthase nucleic acid sequence are mammalian sequences.

7. A composition comprising a vector comprising a cyclooxygenase nucleic acid sequence encoding at least a portion of a coding region of a cyclooxygenase gene, and a prostaglandin synthase nucleic acid sequence encoding at least a portion of a coding region of a prostaglandin synthase gene.

8. The composition of claim 7 wherein the cyclooxygenase gene is cyclooxygenase-1 and the prostaglandin synthase gene is prostaglandin I₂ synthase.

9. The composition of claim 7 wherein the vector is a plasmid.

10. The composition of claim 7 wherein the vector is a viral vector.

11. The composition of claim 10 wherein the vector is a retroviral vector, an adenoassociated vector, an adenoviral vector, or a herpes viral vector.

12. The composition of claim 7 wherein the cyclooxygenase nucleic acid sequence and the prostaglandin synthase nucleic acid sequence are mammalian sequences.

13. A composition comprising a first vector comprising a cyclooxygenase nucleic acid sequence encoding at least a portion of a coding region of a cyclooxygenase gene, and a second vector comprising a prostaglandin synthase nucleic acid sequence encoding at least a portion of a coding region of a prostaglandin synthase gene.

14. The composition of claim 13 wherein the cyclooxygenase gene is cyclooxygenase-1 and the prostaglandin synthase gene is prostaglandin I₂ synthase.

15. The composition of claim 13 wherein the first and second vectors are plasmids.

16. The composition of claim 13 wherein the first and second vectors are viral vectors.

17. The composition of claim 16 wherein the viral vectors are retroviral vectors, adenoassociated vectors, adenoviral vectors, lentiviral vectors, or herpes viral vectors.

18. The composition of claim 13 wherein the cyclooxygenase nucleic acid sequence and the prostaglandin synthase nucleic acid sequence are mammalian sequences.

19. A composition comprising a cyclooxygenase-1 peptide and a prostaglandin I₂ synthase peptide.

20. A method of making a vector, the method comprising the steps of:
a. ligating an expression vector with a cyclooxygenase-1 (COX-1) nucleic acid sequence and a prostaglandin synthase (PGIS) nucleic acid sequence to produce a COX-1-PGIS expression vector.

21. The method of claim 20 wherein said ligating results in the cyclooxygenase-1 nucleic acid and the prostaglandin I₂ synthase nucleic acid each being operatively linked to regulatory sequences directing the expression of said cyclooxygenase-1 and prostaglandin I₂ synthase sequences.

22. The method of claim 20 wherein said expression vector is a plasmid or a viral vector.

23. The method of claim 22 wherein said viral vector is a retroviral vector, an adenoassociated vector, an adenoviral vector, a lentiviral vector, or a herpes viral vector.

24. A method of treating a patient, the method comprising the step of:
a. administering to a patient a composition comprising a vector comprising a cyclooxygenase nucleic acid sequence encoding at least a portion of a coding region of a cyclooxygenase gene, and a prostaglandin synthase nucleic acid sequence encoding at least a portion of a coding region of a prostaglandin synthase gene.

25. The method of claim 24 wherein the cyclooxygenase gene is cyclooxygenase-1 and the prostaglandin synthase gene is prostaglandin I₂ synthase.

26. The method of claim 24 wherein the patient is a human.

27. The method of claim 26 wherein the patient is afflicted with a vascular disorder and wherein the disorder is in any stage of development.

28. The method of claim 27 wherein the vascular disorder is associated with at least one condition selected from the group consisting of stroke, pulmonary hypertension, coronary artery disease, cerebrovascular thrombosis, myocardial infarction, diabetic peripheral vascular disease, and non-diabetic peripheral vascular disease.

29. The method of claim 24 wherein the vector is a retroviral vector, an adenoassociated vector, an adenoviral vector, a lentiviral vector, or a herpes viral vector.

30. The method of claim 24 wherein the composition is administered to at least one targeted site within the patient.

31. The method of claim 30 wherein the at least one targeted site is selected from the group consisting of a cerebral ventricle, a femoral artery, and a coronary artery.

32. The method of claim 31 wherein the at least one condition is a stroke and the at least one targeted site is a cerebral ventricle.

33. A method for treating a patient, the method comprising the steps of:
a. administering to a patient a composition comprising a vector comprising a cyclooxygenase nucleic acid sequence encoding at least a portion of a coding region of a cyclooxygenase gene; and
b. administering to the patient a composition comprising a vector comprising a prostaglandin synthase nucleic acid sequence encoding at least a portion of a coding region of a prostaglandin synthase gene.

34. The method of claim 33 wherein step a is carried out before, after, or at about the same time as step b.

35. The method of claim 33 wherein the vector of steps a and b is a retroviral, an adenoassociated, an adenoviral, a lentiviral vector, or a herpes viral vector.

36. The method of claim 33 wherein the patient is a human.

37. The method of claim 36 wherein the patient is afflicted with a vascular disorder and wherein the disorder is in any stage of development.

38. The method of claim 37 wherein the vascular disorder is associated with at least one condition selected from the group consisting of stroke, pulmonary hypertension, coronary artery disease, cerebrovascular thrombosis, myocardial infarction, diabetic peripheral vascular disease, and non-diabetic peripheral vascular disease.

39. The method of claim 33 wherein the composition is administered to at least one targeted site within the patient.

40. The method of claim 39 wherein the at least one targeted site is selected from the group consisting of a cerebral ventricle, a femoral artery, and a coronary artery.

41. The method of claim 40 wherein the at least one condition is a stroke and the at least one targeted site is a cerebral ventricle.

42. A method of treating a patient, the method comprising the steps of:
a. increasing the level of prostaglandin I₂ in a patient; and
b. increasing the level of cyclooxygenase-1 in a patient,
wherein the level of prostaglandin E₂ in the patient does not increase as a result of steps a and b.

43. The method of claim 42 wherein the patient is a human.

44. The method of claim 42 wherein the patient is afflicted with a vascular disorder and wherein the disorder is in any stage of development.

45. The method of claim 44 wherein the vascular disorder is associated with at least one condition selected from the group consisting of stroke, pulmonary hypertension, coronary artery disease, cerebrovascular thrombosis, myocardial infarction, diabetic peripheral vascular disease, and non-diabetic peripheral vascular disease.

46. The method of claim 42 wherein the composition is administered to at least one targeted site within the patient.

47. The method of claim 46 wherein the at least one targeted site is selected from the group consisting of a cerebral ventricle, a femoral artery, and a coronary artery.

48. The method of claim 47 wherein the at least one condition is a stroke and the at least one targeted site is a cerebral ventricle.

49. A method of treating a patient, the method comprising the steps of:
a. administering to a patient a composition comprising cyclooxygenase-1 and prostaglandin I₂ synthase,
wherein the level of prostaglandin E₂ and the level of prostaglandin F_{2α} in the patient are not increased.

50. The method of claim 49 wherein the patient is a human.

51. The method of claim 50 wherein the patient is afflicted with a vascular disorder and wherein the disorder is in any stage of development.

52. The method of claim 51 wherein the vascular disorder is associated with at least one condition selected from the group consisting of stroke, pulmonary hypertension, coronary artery disease, cerebrovascular thrombosis, myocardial infarction, diabetic peripheral vascular disease, and non-diabetic peripheral vascular disease.

53. The method of claim 49 wherein the composition is administered to at a targeted site within the patient.

54. The method of claim 53 wherein the targeted site is selected from the group consisting of a cerebral ventricle, a femoral artery, and a coronary artery.

55. The method of claim 54 wherein the at least one condition is a stroke and the at least one targeted site is a cerebral ventricle.

56. Any and all methods for treating a patient having a vascular disorder, wherein the method comprises
a. increasing the level of prostaglandin I₂ in a patient;
b. increasing the level of cyclooxygenase-1 in a patient,
wherein the level of prostaglandin E₂ and the level of prostaglandin F_{2α} in the patient are not increased as a result of steps and b.
